SEQUENCE LISTING (17 sequences)

SEQ ID NO: 1 - DNA, 44 bp, Homo sapiens
  misc_feature: human leukocyte antigen E
gggaagcttcgcgacatgggtggtggcggttctggtggtggcgg

SEQ ID NO: 2 - DNA, 44 bp, Homo sapiens
  misc_feature: human leukocyte antigen E
tagtggcggcggaggaagcggtggtggcggttccggagctcggg

SEQ ID NO: 3 - DNA, 24 bp, Homo sapiens
  misc_feature: human leukocyte antigen E
cccgagctccggaaccgccaccac

SEQ ID NO: 4 - DNA, 33 bp, Homo sapiens
  misc_feature: human leukocyte antigen E
gggaagctttgagccgagatgtctcgctccgtg

SEQ ID NO: 5 - DNA, 37 bp, Homo sapiens
  misc_feature: human leukocyte antigen E
cccagcttcagcgtacgccaaagattcaggtttactc

SEQ ID NO: 6 - DNA, 32 bp, Homo sapiens
  misc_feature: human leukocyte antigen E
atgtcgcgatcccacttaactatcttgggctg

SEQ ID NO: 7 - DNA, 31 bp, Homo sapiens
  misc_feature: human leukocyte antigen E
ggttccggatctcactccttgaagtatttcc

SEQ ID NO: 8 - DNA, 32 bp, Homo sapiens
  misc_feature: human leukocyte antigen E
gggtctagattacaagctgtgagactcagacc

SEQ ID NO: 9 - DNA, 64 bp, Homo sapiens
  misc_feature: human leukocyte antigen E
cgcttcctccgccgccactaccgccaccaccagaaccgccaccacccatgtcgcgaagcttccc

SEQ ID NO: 10 - DNA, 69 bp, Homo sapiens
  misc_feature: human leukocyte antigen E
accggatccgccaccacctaaaattaaagtccgcggagccataacagcctcgaggccagaaagagagag

SEQ ID NO: 11 - DNA, 63 bp, Homo sapiens
  misc_feature: human leukocyte antigen E
cccaagcttggcggatccggtggtggcggttctggtggtggcggctccatccagcgtacgcca

SEQ ID NO: 12 - DNA, 63 bp, Homo sapiens
  misc_feature: human leukocyte antigen E
tggcgtacgctggatggagccgccaccaccagaaccgccaccaccggatccgccaagcttggg

SEQ ID NO: 13 - DNA, 1524 bp, Homo sapiens
  misc_feature: human leukocyte antigen E-Single Chain Trimer
aagctttgagccgagatgtctcgctccgtggccttagctgtgctcgcgctactctctctttctggcctcgaggctgttatggctccgcggactttaattttaggtggtggcggatccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactataaggctgagtggagcgacagtgcccaggggtctgagtctcacagcttgtaatctaga

SEQ ID NO: 14 - DNA, 1452 bp, Homo sapiens
  misc_feature: human leukocyte antigen E-Single Chain Dimer
aagctttgagccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactataaggctgagtggagcgacagtgcccaggggtctgagtctcacagcttgtaatctaga

SEQ ID NO: 15 - protein, 500 aa, Homo sapiens
  MISC_FEATURE: human leukocyte antigen E Single chain Trimer
MSASVALAVLALLSLSGLGAVMAPATLILGGGGSGGGGSGGGGSIGATPLIGVTSAHPAGAGLSAPLACTVSGPHPSAIGVALLLAGGAIGLVGHSALSPSLATSPTLLTTTGPTPTGLAGTACAVAHVTLSGPLIVLTAAAMGGGGSGGGGSGGGGSGGGGSGSHSLLTPHTSVSAPGAGGPAPISVGTVAATGPVAPAAAAASPAMVPAAPTMGGGGSGTTAAGTASAAATAGIPAVALATLAGTTAGSGAGSHTLGTMHGCGLGPAAAPLAGTGGPATAGLATLTLAGALASTTAVATAAGISGGLSAAASGAGHGAATLGATCVGTLHLTLGLGLGTLLHLGPPLTHVTHHPISAHGATLACTALGPTPAGITLTTGGAGGGHTGATGLVGTAPAGAGTPGLTAAVVVPSGGGGATTCHVGHGGLPGPVTLATLPASGPTIPIVGIIAGLVLLGSVVSGAVVAAVITALLSSGGLGGSTTLAGTSASAGGSGSHSL

SEQ ID NO: 16 - protein, 476 aa, Homo sapiens
  MISC_FEATURE: human leukocyte antigen E-Single chain dimer
MSASVALAVLALLSLSGLGAIGATPLIGVTSAHPAGAGLSAPLACTVSGPHPSAIGVALLLAGGAIGLVGHSALSPSLATSPTLLTTTGPTPTGLAGTACAVAHVTLSGPLIVLTAAAMGGGGSGGGGSGGGGSGGGGSGSHSLLTPHTSVSAPGAGGPAPISVGTVAATGPVAPAAAAASPAMVPAAPTMGGGGSGTTAAGTASAAATAGIPAVALATLAGTTAGSGAGSHTLGTMHGCGLGPAAAPLAGTGGPATAGLATLTLAGALASTTAVATAAGISGGLSAAASGAGHGAATLGATCVGTLHLTLGLGLGTLLHLGPPLTHVTHHPISAHGATLACTALGPTPAGITLTTGGAGGGHTGATGLVGTAPAGAGTPGLTAAVVVPSGGGGATTCHVGHGGLPGPVTLATLPASGPTIPIVGIIAGLVLLGSVVSGAVVAAVITALLSSGGLGGSTTLAGTSASAGGSGSHSL

SEQ ID NO: 17 - protein, 9 aa, Homo sapiens
  MISC_FEATURE: human leukocyte antigen E
VMAPRTLIL

(19) United States
(12) Patent Application Publication
(10) Pub. No.: US 2004/0225112 A1
(43) Pub. Date: Nov. 11, 2004

(54) Genes encoding single chain human leukocyte antigen E (HLA-E) proteins to prevent natural killer cell-mediated cytotoxicity

(76) Inventor: Mark D. Crew, Little Rock, AR (US)

Correspondence Address: BUTLER, SNOW, O'MARA, STEVENS & CANNADA PLLC, 6075 POPLAR AVENUE, SUITE 500, MEMPHIS, TN, 38119, US

(21) Appl. No.: 10/430,984

(22) Filed: May 6, 2003

(51) Int. Cl. A01K 67/00; C07H 21/04; C07K 14/74; C12P 21/02; C12N 5/06; A61K 38/17

(52) U.S. Cl. 530/350; 800/8; 514/12; 536/23.5; 435/69.1; 435/320.1; 435/325

(57) ABSTRACT

Single chain polypeptide forms of HLA-E useful in manipulating and ascertaining natural killer (NK) cell function are disclosed. The single chain trimer (SCT) form of HLA-E is comprised of the signal peptide from human beta-2 microglobulin (β2m), a canonical HLA-E binding peptide, a fifteen amino acid linker, mature human β2m, a twenty amino acid linker, and mature HLA-E heavy chain. The single chain dimer (SCD) form of HLA-E is comprised of the signal peptide from human β2m, mature human β2m, a twenty amino acid linker, and mature HLA-E heavy chain. The disclosed polypeptides can be used to inhibit NK cell cytotoxicity and cytokine production, enumerate and/or purify NK cell subsets, and identify biologically relevant HLA-E ligands. The disclosed HLA-E SCT and SCD nucleic acid sequences can be used a platform for synthesis of additional biologically active major histocompatibility class I protein single chain trimers and dimers.

### STATEMENT REGARDING FEDERALLY SPONSORED RESEARCH OR DEVELOPMENT

The invention was made with Government support under the terms of A149885 awarded by NIH/NIAID and the Office of Research and Development, Department of Veterans Affairs. The Government has certain rights in the invention.

### CROSS-REFERENCE TO RELATED APPLICATIONS

Not applicable.

### BACKGROUND OF THE INVENTION

1. Field of the Invention

The present invention relates to genetic technology to eliminate human natural killer (NK) cell-mediated rejection of xenografts.

2. Brief Description of the Related Art

Pig-to-human xenotransplantation is an attractive means to alleviate the critical shortage of human organs. Human natural killer (NK) cells, although not generally considered significant in allotransplants, may play an important role in the rejection of porcine xenografts.

Several lines of evidence suggest that NK cells participate in pig-to-primate xenograft rejection. First, there are numerous reports describing the killing of cultured pig cells by human NK cells (Seebach et al. 1996; Chan & Auchincloss, 1996; Donnelly et al. 1997; Matter-Reissmann et al. 2002, Xenotransplantation 9:325-337; Horvath-Arcidiacono & Bloom; 2003). Second, recipient NK cell infiltration has been observed in pig kidney grafts undergoing acute vascular rejection (AVR) in a pig-to-cynomolgus monkey model and more infiltrating NK cells were observed in grafts undergoing AVR than in grafts without AVR (Quan et al. 2000). Finally, human NK infiltration of pig kidneys is also seen in an ex vivo perfusion model using human blood (Khalfoun et al., Surgery 128:447-457).

NK cells are a key component of the innate immune system and influence adaptive immune responses via cytokine secretion. The activity of NK cells is thought to be controlled by the balance of inhibitory and activating signals delivered via NK cell cell-surface receptors (Lopez-Botet & Bellon 1999; Curr Opin Immunol. 11:301-307). Conceivably then, eliminating ligands for NK cell activation receptors on pig cells or increasing the level of ligands for inhibitory cell receptors could abrogate human NK cell-mediated destruction of porcine xenografts. The latter strategy has received the most attention probably due to the scant understanding of porcine NK cell activating ligands.

There are two classes of NK cell inhibitory receptors: the immunoglobulin-like KIR and LIR receptors and the C-type lectin-like receptors (CD94/NKG2 heterodimers). In humans, the ligands for the KIR receptor family members are the classical class I antigens, HLA-A, -B, and -C and the ligand for some LIRs (LIR-1 and -2) is the nonclassical class I antigen HLA-G. The major ligand for CD94/NKG2 receptors is the nonclassical class I antigen HLA-E. Ligands for several types of human NK cell inhibitory receptors have been expressed in pig cells and tested for their ability to modulate NK cell activity.

When ligands for KIRs, specifically HLA-A2, -B27, and -Cw3, were expressed in immortalized porcine endothelial cells, only HLA-Cw3 conferred protection against lysis by human NK cells but only if the NK cells expressed CD158b; protection against lysis by NK cells expressing CD158a was not observed (Seebach et al. 1997; J Immunol. 159:3655-3661). Utilization of a classical class I antigen such as HLA-Cw3 is problematic insofar as the induction of alloreactive T cells may occur. Mutation of the CD8 binding site of HLA-Cw3 (D227K) ameliorated its potential alloreactivity but consistent with the results of Seebach et al. (1997; J Immunol. 159:3655-3661), complete protection to lysis by CD158b+ NK cells but only partial protection against lysis by polyclonal NK cell preparations was observed (Sharland et al., 2002, J Immunol. 168:3266-3274).

HLA-G has been explored as a potential inhibitor of human NK cell lysis of pig cells with mixed results. An early report describes dramatic decreases in the ability of human NK cells to lyse porcine aortic endothelial cells transfected with HLA-G (Sasaki et al. 1999; Transplantation 67:31-37). However, results from other studies suggest that HLA-G either only partially protects against human NK cell-mediated cytotoxicity (Forte et al., 2001, J Immunol. 167:6002-6008; Matsunami et al. 2002; Transplantation 73:1582-1589) or fails completely (Dorling et al., 2000, Eur J Immunol. 30:586-593) although human NK cell/porcine endothelial cell interaction may be appreciably altered (Dorling et al., 2000, Eur J Immunol. 30:586-593; Forte et al., 2001, J Immunol. 167:6002-6008).

Among NK cell inhibitory receptors, CD94/NKG2A appears to be widely expressed among NK cells. Thus, the ligand for CD94/NKG2A, HLA-E, when expressed on porcine cells might be the most potent inhibitor of human NK cell lysis. The cell-surface expression of HLA-E on pig cells is somewhat controversial. Sasaki et al. (1999; J Immunol. 163:6301-6305) report that transfection of the HLA-E gene together with the human β2-microglobulin (β2m) gene resulted in readily detectable cell-surface expression of HLA-E and conferred a 34-84% reduction in NK cell-mediated killing of porcine endothelial cells. Matsunami et al. (2002; Transplantation 73:1582-1589), on the other hand, detected HLA-E cell-surface expression on transfected porcine endothelial cells only when a canonical HLA-E binding peptide was endogenously added. The canonical HLA-E binding peptide is found in the leader peptide of HLA-A, -B, -C, and -G proteins (Braud et al., 1998, Curr Biol. 8:1-10) and HLA-E expression was also detected when the HLA-E gene was co-transfected with the HLA-G gene or when the leader peptide-encoding sequence of HLA-E was replaced with the corresponding sequences of HLA-A2 or HLA-G. The discrepancy regarding cell-surface expression of HLA-E might be due to the difference in the strains of pigs from which the endothelial cells were derived. That is, an HLA-E binding peptide may be expressed in one strain but not another. Pig strains expressing an HLA-E binding peptide might be quite rare as cell-surface of expression of transfected HLA-E was not observed in three additional, independently-derived porcine cell lines (M.D.C, unpublished observations).

The binding of HLA-E to CD94/NKG2A, and subsequent negative signaling is highly dependent on the nature of the peptide bound to HLA-E and the HLA class I signal sequence-derived peptides are optimal in this regards. Although not rigorously examined, human β2m may also be required for maximal cell-surface expression in pig cells. Generating pigs transgenic for three genes (HLA-E heavy chain, human β2m, and some gene encoding an HLA-E binding peptide) in order to ensure HLA-E cell-surface expression is technically difficult and would be tedious. While the leader peptide of HLA-E could be replaced by one containing a canonical HLA-E binding peptide, the level of peptide produced may not be sufficient to keep HLA-E bound solely with that peptide.

Yu et al. described a single chain trimer of a mouse classical class I protein (H-2 K^{b}) in which the peptide antigen (“OVA”) bound to the heavy chain is covalently attached by a fifteen amino acid peptide linker to mouse β2m which is itself attached to the Kb heavy chain by a twenty amino acid peptide linker (Yu et al., 2002, J Immunol. 168:3145-3149). The OVA peptide was shown to be extraordinarily tightly bound to the K^{b} heavy chain. Moreover, the single chain OVA-β2m-K^{b} trimer was able to induce OVA-specific, K^{b}-restricted T cell responses.

There exits a need in the art to circumvent having to separately express human β2m and an HLA-E binding peptide in order to achieve HLA-E cell-surface expression. The invention herein describes the construction of a single chain trimer (SCT) of HLA-E. Furthermore, the invention also describes the expression and functional analysis of the HLA-E SCT in pig cells.

References mentioned in this background section are not admitted to be prior art with respect to the present invention.

### BRIEF SUMMARY OF THE INVENTION

Single chain polypeptide forms of HLA-E useful in manipulating and ascertaining natural killer (NK) cell function are disclosed. The single chain trimer (SCT) form of HLA-E is comprised of the signal peptide from human beta-2 microglobulin (β2m), a canonical HLA-E binding peptide, a fifteen amino acid linker, mature human β2m, a twenty amino acid linker, and mature HLA-E heavy chain. The single chain dimer (SCD) form of HLA-E is comprised of the signal peptide from human β2m, mature human β2m, a twenty amino acid linker, and mature HLA-E heavy chain. The disclosed polypeptides can be used to inhibit NK cell cytotoxicity and cytokine production, enumerate and/or purify NK cell subsets, and identify biologically relevant HLA-E ligands. The disclosed HLA-E SCT and SCD nucleic acid sequences can be used a platform for synthesis of additional biologically active major histocompatibility class I protein single chain trimers and dimers.

### BRIEF DESCRIPTION OF THE DRAWINGS

These and other features, objects and advantages of the present invention will become better understood from a consideration of the following detailed description and accompanying drawings.

FIG. 1 shows a schematic of HLA-E SCT and SCD structures. Human β2m-encoding sequences, including those encoding the signal peptide (s.p.), are shown in gray. HLA-E sequences are shown in black and the peptide antigen-encoding region of HLA-E SCT is white. The positions and composition of connecting peptides are given above each construct.

FIG. 2 shows cell-surface expression of the 3D12 (HLA-E) epitope in transiently transfected LLC-PK1 cells. The 3D12 staining of EGFP-positive cells is shown with the dashed curve from vector-transfected LLC-PK1 cells, gray curve from HLA-E SCD transfectants, and solid black from HLA-E SCT transfectants. Note the increased mean fluorescent intensity in HLA-E SCT transfectants relative to HLA-E SCD and vector transfectants

FIG. 3 shows flow cytometric analyses of LLC-PK1 cell stable transfectants. 3D12 staining of untransfected LLC-PK1 cells and LLC-PK1 cells transfected with HLA-E SCD or HLA-E SCT (as noted above each histogram) is shown. Note that 3D12 positive cells have a higher mean fluorescent intensity in HLA-E SCT transfectants compared to HLA-E SCD transfectants.

FIG. 4 shows flow cytometric analyses of sorted HLA-E SCT LLC-PK1 cell stable transfectants. Untransfected LLC-PK1 cells (open curves) or HLA-E SCT transfected LLC-PK1 cells which were sorted based on 3D12 staining (filled curves) were analyzed by flow cytometry using the mAbs listed above each histogram.

FIG. 5 shows NK cell-mediated lysis of untransfected and HLA-E SCT transfected LLC-PK1 cells. Shown are the percent specific lysis of untransfected LLC-PK1 cells (filled circles) and HLA-E SCT transfected LLC-PK1 cells (open circles) at 4 hours at various effector:target ratios (upper graphs) and at an effector:target ratio of 10:1 for various times (lower graphs) by NK92 or NKL cells (as indicated above the graphs). Note the decreased susceptibility to NK cell-mediated lysis conferred by HLA-E SCT.

FIG. 6 shows γ-interferon secretion by co-cultured NKL cells. The concentration of γ-interferon in supernatants of LLC-PK1 cells alone (“LLCPK1”), LLC-PK1 cells co-cultured with NKL cells (“LLCPK1+NKL”), HLA-E SCT transfected LLC-PK1 cells co-cultured with NKL cells (“E−SCT+NKL”), and NKL cells cultured alone (“NKL”) is shown. Note the inhibition of γ-interferon imparted by HLA-E SCT.

### DETAILED DESCRIPTION OF THE INVENTION

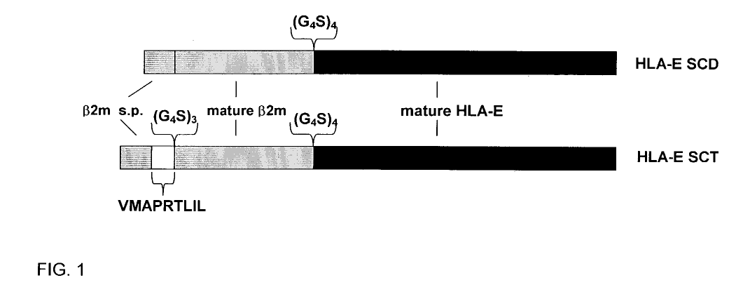

With reference to FIGS. 1-6, the preferred embodiment of the present invention may be described. The present invention is directed to satisfying the need for a single chain trimer gene which folds properly and confers protection against human NK-cell mediated killing.

Structure of HLA-E Single Chain Trimer and Dimer Genes.

The HLA-E single chain trimer (SCT) gene was constructed using standard techniques. As a control, a gene encoding a single chain dimer (SCD) of HLA-E (lacking sequences encoding peptide antigen) was constructed as well. Schematic depiction of HLA-E SCT and SCD genes are shown in FIG. 1. HLA-E SCT consists of the signal peptide-encoding portion of the human β2m gene followed by a sequence encoding a canonical HLA-E binding peptide antigen, VMAPRTLIL which is identical to that found in the signal peptide of HLA-Cw3. The peptide antigen encoding-sequence is followed by a 45 base pair (bp) sequence encoding “connecting peptide 1” which when translated will yield the 15 amino acid sequence (G₄S)₃. Immediately 3′ to connecting peptide 1-encoding DNA is the sequence for mature (lacking signal peptide) human β2m cDNA which is linked to the sequence of mature HLA-E heavy chain by a 60 bp sequence encoding “connecting peptide 2” which when translated will yield the 20 amino acid sequence (G₄S)₄. HLA-E SCD is identical to HLA-E SCT except that the HLA-E SCD gene lacks the peptide antigen- and connecting peptide 1-encoding sequences. The nucleotide sequences of HLA-E SCT and SCD genes have been deposited in GenBank accession numbers HLAESCT AY289236 and HLAESCD AY289237, respectively.

HLA-E SCT and SCD genes are both under control of the CMV immediate early gene promoter so relatively high levels of HLA-E and SCD gene transcripts can be produced in all cell-types. To permit facile swapping of peptide antigen, unique restriction sites (XhoI and BamHI) flank the VMAPRTLIL-encoding sequence of the HLA-E SCT gene. Likewise, the HLA-E heavy chain-encoding domain can easily be replaced by another MHC class I heavy chain gene sequence by using the unique restriction sites, BspEI and XbaI. The HLA-E SCT gene construct therefore provides a platform for the easy and rapid development of other MHC class I single chain trimers.

Cell-Surface Expression of HLA-E SCT and SCD Proteins.

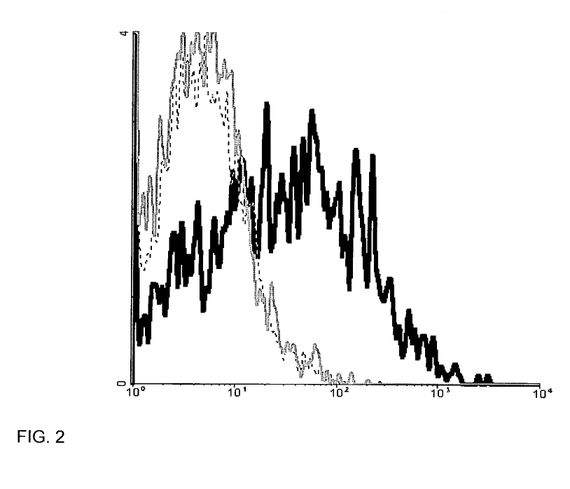

Initial assessment of HLA-E SCT and SCD cell-surface expression utilized transiently transfected LLC-PK1 cells. To identify transfected cells, HLA-E SCT and SCD genes were co-transfected with pEGFP-C1 which encodes the enhanced green fluorescence protein. Forty eight hours post-transfection, cells were harvested, stained with the HLA-E-specific mAb 3D12 and analyzed by flow cytometry, with gating on EGFP positive cells. The results are shown in FIG. 2. Significant 3D12 staining, indicative of HLA-E cell-surface expression, was observed in HLA-E SCT transfectants while HLA-E SCD transfectants exhibited 3D12 staining comparable to that seen in LLC-PK1 cells transfected with just vector. These findings suggest that the covalently attached peptide antigen of HLA-E SCT markedly enhances cell-surface expression and that the HLA-E SCT seems to fold properly.

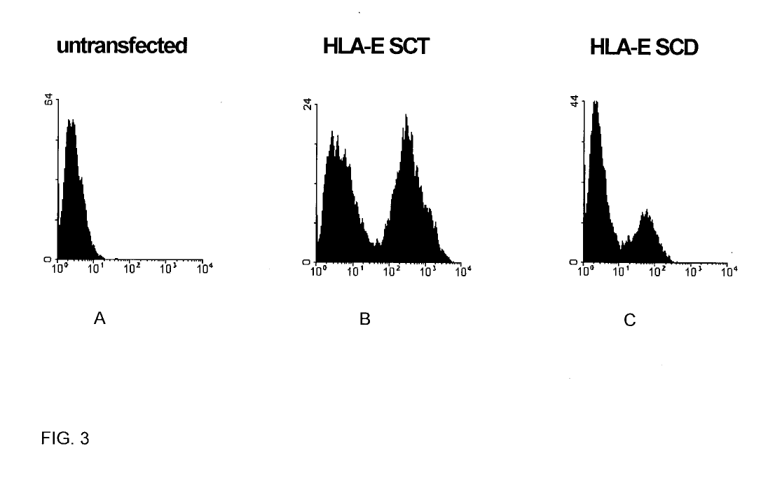

LLC-PK1 cells were stably transfected with HLA-E SCT and SCD and examined for HLA-E expression, again using the HLA-E specific mAb 3D12. Substantial HLA-E cell-surface expression was observed in HLA-E SCT transfectants while no 3D12 staining was observed in LLC-PK1 cells stably transfected with vector alone. As is typical of populations of cells selected for resistance to G418, not all G418 resistant cells were positive for 3D12 staining as is shown in FIG. 3. LLC-PK1 cells stable transfected with HLA-E SCD, unlike those transiently transfected with this construct, showed appreciable levels of HLA-E cell-surface expression. However, the mean fluorescent intensity (MFI) of 3D12 staining was noticeably reduced compared to HLA-E SCT stable transfectants (FIG. 3; MFI of 62 versus 375, respectively). Such results are consistent with the idea that the covalently attached peptide antigen in HLA-SCT significantly increases the stability of the HLA-E SCT.

The reduced cell-surface levels of HLA-E SCD compared to HLA-E SCT that is observed in both transient and stable transfectants is likely due to a paucity of peptides in pig cells capable of binding HLA-E, consistent with the results of Matsunami et al. (2002; Transplantation 73:1582-1589). Peptide-free MHC class I proteins are inherently unstable. This reduced stability of HLA-E SCD actually has great utility as it provides a system to systematically identify HLA-E binding peptides and peptide mimics. That is, HLA-E binding peptides when added exogenously to LLC-PK1 HLA-E SCD stable transfectants would increase 3D12 staining in an amount proportional to the affinity of the peptide to HLA-E SCD.

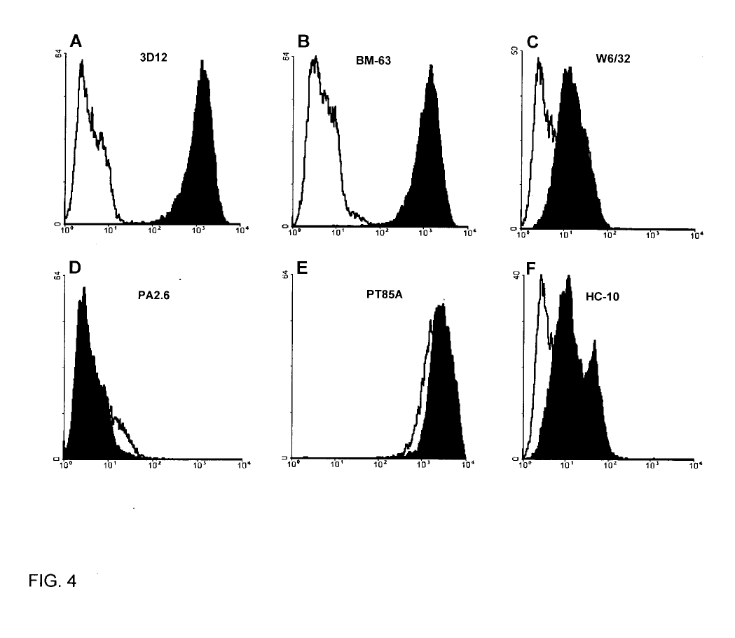

A homogenous population of HLA-E SCT positive LLC-PK1 cells was obtained by fluorescent activated cell sorting using mAb 3D12. These were analyzed by flow cytometry with an expanded panel of specific mAbs (FIG. 4). The cell sorting was effective and efficient in that 100% of the cells were stained with the HLA-E specific mAb 3D12 (FIG. 4A). The cells were also all positive for BM-63 (FIG. 4B), a mAb specific for human β2m. mAb BM-63 is not only human-specific but its binding is also conformational dependent; the high MFI observed thus indicated that at least the β2m domain of HLA-E SCT is folded correctly. Two pan-HLA class I-specific mAbs, W6/32 and PA2.6, both of which recognize HLA-E, were also tested (FIG. 4C and D, respectively). HLA-E SCT transfected LLC-PK1 cells were uniformly positive for W6/32 although the fluorescent intensity was quite weak. The weak staining by W6/32 can be attributed to the fact that the epitope of W6/32 includes the amino terminus of human β2m (Shields and Ribaudo, Tissue Antigens. 51:567-570) which is not present in HLA-E SCT. HLA-E SCT expressing LLC-PK1 cells were negative for PA2.6 (FIG. 4D). The epitope of PA2.6 maybe conformation dependent and such conformation is lost by inclusion of the connecting peptides in HLA-E SCT. Alternatively, the connecting peptides may mask an amino acid(s) that is part of the PA2.6 epitope. PT85A is a conformation dependent mAb purportedly specific to porcine MHC class I antigens but also binds at least some HLA class I antigens (M.D.C. unpublished observations). PT85A stained brightly untransfected LLC-PK1 cells; staining of HLA-E SCT transfected cells was slightly, but reproducibly, higher (FIG. 4E). mAb HC-10 was used to gauge the level of misfolded or β2m-free HLA-E heavy chain. HC-10 staining was slightly higher in HLA-E SCT transfectants compared to untransfected LLC-PK1 cells and in some cases (like that shown in FIG. 4F) a bimodal distribution was observed. These results suggest that perhaps a small fraction of HLA-E SCT is not folded correctly or the β2m moiety was proteolytically damaged. Taken together, the flow cytometric analyses of HLA-E SCT-expressing LLC-PK1 cells indicate that the vast majority of HLA-E SCT expressed on the cell-surface is serologically undistinguishable from correctly folded, native HLA-E.

Susceptibility of Pig (LLC-PK1 ) Cells Expressing HLA-E SCT to Lysis by Human NK Cells.

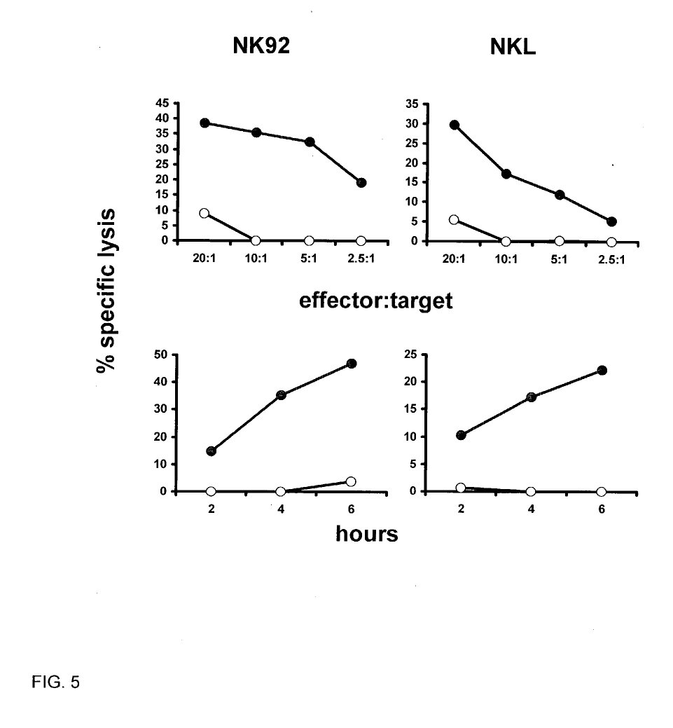

Flow cytometric analyses suggested that HLA-E SCT is expressed at the cell-surface with a correct conformation (FIGS. 2-4) but such analyses do not demonstrate that it is functional. The functionality of HLA-E SCT was directly assessed by testing its ability to confer protection against human NK cell-mediated lysis. Two NK cell lines, NK-92 and NKL, were used as effectors in standard ⁵¹Cr-release assays to quantify cytotoxicity. As targets, untransfected LLC-PK1 cells or LLC-PK1 cells transfected with HLA-E SCT were used. The results, shown in FIG. 5, clearly demonstrate that HLA-E SCT protects LLC-PK1 cells from killing by human NK cells. Untransfected LLC-PK1 cells were specifically lysed by NK92 cells at effector:target ratios ranging from 2.5:1 to 20:1 in a time-dependent manner (FIG. 5). In contrast, LLC-PK1 cells expressing HLA-E SCT were almost completely protected with only minimal lysis observed at 6 hours or at an effector:target ratio of 20:1 (FIG. 5). NKL cells lysed untransfected LLC-PK1 cells to a slightly lesser degree than did NK92 cells but the results with regards to HLA-E SCT were identical—the susceptibility to lysis was virtually abolished by expression of HLA-E SCT (FIG. 5). Thus, HLA-E SCT, in which all three components of a normal HLA-E protein complex (heavy chain, β2m, and peptide) are in one polypeptide chain, is immunologically functional in terms of its ability to modulate NK cell cytotoxicity. Organs from pigs which are transgenic for HLA-E SCT would not be subject to NK cell-mediated rejection, at least not by CD94/NKG2A-positive human NK cells (which generally comprise 75-90% of peripheral blood NK cells).

Reduced γ-IFN Secretion of Human NK Cells in Response to Pig (LLC-PK1) Cells Expressing HLA-E SCT.

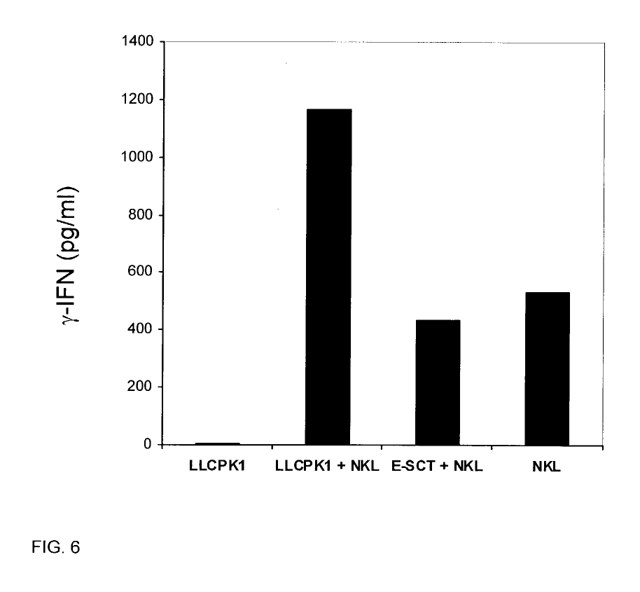

NK cells participate in the innate immune response not only by their cytolytic activity but also by their secretion of cytokines which can attract and activate other cells of the innate and adaptive immune systems. The ability of HLA-E SCT to alter NK cell cytokine secretion was therefore examined using a CBA assay which simultaneously measures six different cytokines (IL-2, IL-4, IL-5, IL-10, Tumor Necrosis Factor α, and γ-interferon) in cell culture supernatants. NKL cells were cultured alone or co-cultured with untransfected LLC-PK1 cells or LLC-PK1 cells expressing HLA-E SCT. LLC-PK1 cells by themselves served as a negative control. After 48 hours of co-culture, supernatants were collected and assayed. Of the six cytokines assayed, only γ-interferon is routinely reported as being synthesized and secreted by NK cells and indeed γ-interferon was the only one detectable in the supernatants. NKL cells co-cultured with untransfected LLC-PK1 cells secreted more than two-fold more γ-interferon than NKL cells cultured alone (FIG. 6). Remarkably, γ-interferon secretion by NKL cells co-cultured with LLC-PK1 cells expressing HLA-E SCT was equivalent to that observed with NKL cells alone (FIG. 6). Thus, not only does HLA-E SCT inhibit the cytolytic activity of human NK cells to pig cells, but also prohibits human NK cell cytokine secretion incurred by contact with pig cells. The downstream events following cytokine secretion by human NK cells may have more dramatic consequences in xenotransplantation than actual NK cell-mediated lysis. The ability of HLA-E SCT to inhibit NK cell cytokine secretion complements its ability to inhibit NK cell-mediated cytotoxicity.

### EXAMPLES

### Example 1

### Cell Lines and Monoclonal Antibodies (mAbs)

The pig kidney epithelial cell line, LLC-PK1, and the human NK cell line, NK-92 were obtained from American Type Culture Collection (ATCC, Manassas, Va., USA). The human NK cell line, NKL, was a gift from Dr. Michael J. Robertson (Indiana University Medical Center). LLC-PK1 and NK-92 cells were maintained in and maintained in RPMI 1640 supplemented with 10% fetal calf serum, 100 ug/ml penicillin G, and 100 ug/ml streptomycin sulfate (RPMI/10%). NKL cells were propagated in the same except with 15% fetal calf serum and with 200 U/ml IL-2. IL-2 was obtained through the AIDS Research and Reference Reagent Program, Division of AIDS, NIAID, NIH from Dr. Maurice Gately, Hoffman-La Roche Inc.

The mAb PT85A which recognizes a monomorphic determinant of porcine MHC class I proteins (Davis et al. Vet Immunol Immunopathol; 15: 337) was purchased from VMRD, Inc. (Pullman, Wash. USA). mAb BM-63 which is specific for human β2m was purchased from Sigma (St. Louis, Mo., USA). The HLA-E-specific mAb, 3D12, was kindly provided by Dr. Daniel Geraghty (Fred Hutchinson Cancer Research Center, Seattle Wash., USA). The pan-HLA class I mAbs w6/32 and PA2.6 were obtained from ascites. HC-10 mAb which recognizes β2m-free\misfolded HLA class I heavy chain was provided by Dr. Ted Hansen (Washington University, St. Louis, Mo., USA). In some flow cytometric analyses, mAb UPC10 was used as isotype control (IgG₂ₐ, kappa) and PE-conjugated goat anti-mouse IgG was employed as a secondary antibody; both were purchased from Sigma (St. Louis, Mo., USA).

### Example 2

### Construction of HLA-E SCT Gene

To construct a gene encoding an HLA-E single chain trimer (SCT), DNA fragments encoding the β2m leader peptide linked to the VMAPRTLIL peptide, mature β2m, connecting peptide 1, and connecting peptide 2 were individually cloned into plasmids. These fragments were sequentially ligated together and subsequently fused to sequences encoding the mature HLA-E heavy chain. Oligonucleotides used in the construction of HLA-E SCT are given in the Sequence Listing.

The plasmid pB2MLP-pep contains a fragment encoding the β2m leader peptide linked to the VMAPRTLIL peptide. pB2MLP-pep was constructed by PCR amplification using the primers designated B2MF and B2MR with cloned full length human β2m cDNA as template. The PCR product was digested with BamHI and HindIII and ligated into BamHI- and HindIII-cleaved pBluescript-SK+ (Stratagene, La Jolla, Calif., USA).

pMB which contains a DNA fragment encoding mature β2m was derived from PCR amplification using primers B2MF2 and B2MR2 with cloned full length human β2m cDNA as template. The PCR product was ligated directly into pCR2.1 (Invitrogen).

pC1 contains a fragment encoding connecting peptide 1 and was derived by annealing oligonucleotides C1F and C1R and ligating the resulting double stranded DNA into EcoRV-cleaved pBluescript-SK+. pC2 contains a fragment encoding connecting peptide 2 and was made by annealing oligonucleotides C2F1, C2F2, C2R1, and C2R1, cutting the resulting double stranded DNA with HindIII and SacI followed by ligation into HindIII- and SacI-cleaved pBluescript-SK+.

The insert of pC1 was cloned into pMB2M using BsiWI and XhoI to generate pC1-MB. The insert of pC1-MB was cloned into pC2 using HindIII and NruI to create pC1-MB-C2. The insert of pC1-MB-C2 was cloned into pB2mLP-pep using BamHI and SacI to create pLPpep-C1-MB-C2.

The final steps in the construction of the HLA-E SCT gene began with PCR amplification of mature HLA-E heavy chain-encoding sequences using HLAEF and HLAER primers with cloned full length HLA-E cDNA as template. The PCR product was digested with BspEI and XbaI and ligated with the insert of pLPpep-C1-MB-C2, excised using HindIII and BspEI, into HindIII- and XbaI-cleaved pcDNA3.1 (ClonTech, Pal Alto, Calif., USA). The HLA-E SCT gene is thus downstream of the CMV promoter and contains at its 3′ end an SV40-derived polyadenylation signal.

### Example 3

### Construction of HLA-E SCD Gene

A gene encoding an HLA-E single chain dimer (SCD), i.e. encoding the HLA-E heavy chain linked to β2m, including its leader peptide, was constructed by PCR amplification of the cloned human β2m gene using B2MF and B2MR2 primers. The resulting PCR product was digested with HindIII and EcoRI (which cleaves within the mature β2m coding sequence) and ligated in place of the HindIII, EcoRI fragment of HLA-E SCT.

### Example 4

### Transfection of LLC-PK1 Cells

LLC-PK1 cells were transiently and stably transfected. For transient transfections, 3×10⁵ cells were plated in 10 mm plates and allowed to adhere overnight at 37 C in RPMI/10%. Two hours before transfection, the medium was replaced with 600 ul OptiMEM (Life Technologies, Gaithersburg, Md., USA). To identify transiently transfected cells, HLA-E SCT or HLA-E SCD constructs (4 ug each) were co-transfected with 2 ug pEGFP-C1 (Clontech). Plasmids were resuspended in 200 ul OptiMEM and mixed with 200 ul OptiMEM with 10 ul Lipofectamine (Life Technologies). After 20 minutes at room temperature, DNA/Lipofectamine mixtures were added directly to the cells (final volume of 1 ml). Cells were harvested 48 hours after transfection for flow cytometric analyses as described below.

LLC-PK1 cells were stably transfected by electroporation. In brief, 2×10⁶ LLC-PK1 cells were resuspended in 200 ul RPMI/10% to which was added 20 ug DNA in 200 ul RPMI/10%. Electroporation was performed at 250 V, 960 uF and cells were replated in 5 ml RPMI/10%. Two days later, G418 was added to a final concentration of 1 mg/ml.

### Example 5

### Flow Cytometry

LLC-PK1 transfectants, removed from plates by trypsinization, were washed once with wash buffer (phosphate buffered saline, PBS, with 2% fetal calf serum and 0.2% NaN₃) and incubated on ice for 30-60 minutes with saturating concentrations of primary antibody. Cells were washed twice with wash buffer to remove unbound antibody. When PT85A was used as primary antibody, the cells were subsequently incubated with PE-conjugated goat anti-mouse IgG for 30-60 minutes on ice in wash buffer. Prior to flow cytometry all cells were fixed in PBS containing 1% paraformaldehyde. Flow cytometric analyses were performed using the FACSCalibur instrument (Becton Dickinson, Franklin Lakes, N.J. USA).

### Example 6

### Cytotoxicity Assays

NK cell cytotoxicity was measured by standard ⁵¹Cr release assays with either NK-92 or NKL cells as effectors. Confluent monolayers of target cells, LLC-PK1 cells or LLC-PK1 HLA-E SCT transfectants, were incubated in RPMI/10% with 10 uCi/ml ⁵¹Cr for 16 hours at 37 C. The monolayers were washed three times with PBS prior to trypsinization. Cytotoxicity assays were performed in triplicate in 96 well U-bottom dishes using 10⁴ target cells/well at an effector:target ratios ranging from 20:1 to 2.5:1 in a final volume of 200 ul. After various times of incubation at 37 C (2, 4, or 6 hours), 25 ul of supernatant was removed and the radioactivity counted using a Packard gamma counter. Percent specific lysis was calculated using the formula:

cpmₑₓₚₑᵣᵢₘₑₙₜₐₗ−cpmₛₚₒₙₜₐₙₑₒᵤₛ/cpmₘₐₓᵢₘᵤₘ−cpmₛₚₒₙₜₐₙₑₒᵤₛ×100

### Example 7

### Cytokine Measurements

Equal numbers (10⁵ each) of NKL cells and untransfected or HLA-E SCT-transfected LLC-PK1 cells were co-cultured in 200 ul RPMI/10% with 100 U/ml IL-2 for 48 hours at which time 100 ul supernatant was removed. Cytokine (IL-2, IL-4, IL-5, IL-10, Tumor Necrosis Factor α, and γ-interferon) levels in the supernatants were quantified using the BD Human Th1/Th2 Cytokine Cytometric Bead Array™ kit according to the protocol recommended by the supplier (BD Biosciences Pharmingen, San Diego, Calif., USA).

### Sequence Listing

The nucleic and amino acid sequences listed in the accompanying sequence listing are shown using standard letter abbreviations for nucleotide bases. Only one strand of each nucleic acid sequence is shown, but the complementary strand is understood as included by any reference to the displayed strand.

SEQ ID NO. 1 shows the oligonucleotide C2F1 used in the construction of HLA-E single chain trimer.

SEQ ID NO. 2 shows the oligonucleotide C2F2 used in the construction of HLA-E single chain trimer.

SEQ ID NO. 3 shows the oligonucleotide C2R2 used in the construction of HLA-E single chain trimer.

SEQ ID NO. 4 shows the oligonucleotide B2MF used in the construction of HLA-E single chain dimer and trimer.

SEQ ID NO. 5 shows the oligonucleotide B2MF2 used in the construction of HLA-E single chain trimer.

SEQ ID NO. 6 shows the oligonucleotide B2MR2 used in the construction of HLA-E single chain dimer and trimer.

SEQ ID NO. 7 shows the oligonucleotide HLAEF used in the construction of HLA-E single chain trimer.

SEQ ID NO. 8 shows the oligonucleotide HLAER used in the construction of HLA-E single chain trimer.

SEQ ID NO. 9 shows the oligonucleotide C2R1 used in the construction of HLA-E single chain trimer.

SEQ ID NO. 10 shows the oligonucleotide B2MR used in the construction of HLA-E single chain trimer.

SEQ ID NO. 11 shows the oligonucleotide C1F used in the construction of HLA-E single chain trimer.

SEQ ID NO. 12 shows the oligonucleotide C1R used in the construction of HLA-E single chain trimer.

SEQ ID NO. 13 shows the nucleotide sequence of the HLA-E single chain trimer gene.

SEQ ID NO. 14 shows the nucleotide sequence of the HLA-E single chain dimer gene.

SEQ ID NO. 15 shows the amino acid sequence of the HLA-E single chain trimer protein.

SEQ ID NO. 16 shows the amino acid sequence of the HLA-E single chain dimer protein.

What is claimed is:

1. An isolated and purified HLA-E protein comprising the amino acid sequence of SEQ ID NO. 15 that retains biological activity.

2. A composition comprising the protein of claim 1.

3. An isolated HLA-E protein of claim 1 having a configuration to optimize the biological function and three dimensional conformation of said protein.

4. An isolated and purified HLA-E protein comprising the amino acid sequence of SEQ ID NO. 16 that retains biological activity.

5. A composition comprising said protein of claim 4.

6. An isolated HLA-E protein of claim 4 having a configuration to optimize the biological function and three dimensional conformation of said protein.

7. An isolated polynucleotide molecule having a sequence selected from the group consisting of:
(a) a nucleotide sequences shown in SEQ ID NO. 13;
(b) a complete complementary strand of a nucleotide sequence shown in SEQ ID NO. 13; or
(c) fragments of a nucleotide sequence shown in SEQ ID NO. 13.

8. An expression vector containing said polynucleotides of claim 7.

9. A host cell containing said expression vector of claim 8.

10. A transgenic organism having said host cell of claim 9.

11. An isolated polynucleotide molecule having a sequence selected from the group consisting of:
(a) the nucleotide sequences shown in SEQ ID NO. 14;
(b) a complete complementary strand of a nucleotide sequence shown in SEQ ID NO. 14; or
(c) fragments of a nucleotide sequence shown in SEQ ID NO. 14.

12. An expression vector containing said polynucleotides of claim 11.

13. A host cell containing said expression vector of claim 12.

14. A transgenic organism containing said host cell of claim 13.

15. A method for inhibiting natural killer cell-mediated lysis comprising the steps of:
(a) providing a polypeptide sequence of claim 1,
(b) administering to cells or whole animals said polypeptide sequence of claim 1; and
(c) producing an inhibitory response to said natural killer cell-mediated lysis.

16. A method for inhibiting natural killer cell-mediated lysis comprising the steps of:
(a) introducing into a host cell a nucleotide sequence of claim 7,
(b) expressing said nucleotide sequence in said host cell; and
(c) producing an inhibitory response to said natural killer cell-mediated lysis.

17. A method for inhibiting natural killer cell cytokine secretion comprising the steps of:
(a) providing a polypeptide sequence of claim 1,
(b) administering to cells or whole animals said polypeptide sequence of claim 1; and
(c) producing an inhibitory response to said natural killer cytokine secretion.

18. A method for inhibiting natural killer cell cytokine secretion comprising the steps of:
(a) introducing into a host cell a nucleotide sequence of claim 7,
(b) expressing said nucleotide sequence in said host cell; and
(c) producing an inhibitory response to said natural killer cell cytokine secretion.

19. A method to identify a ligand which specifically binds to an HLA-E protein comprising the steps of:
(a) combining a polypeptide sequence of claim 4, with a plurality of molecules, under conditions to allow specific binding,
(b) screening said plurality of molecules, and
(c) detecting specific binding, thereby identifying a ligand that specifically binds to said HLA-E protein.

20. A method to identify a ligand which specifically binds to an HLA-E protein comprising the steps of:
(a) combining a cell of claim 13, with a plurality of molecules, under conditions to allow specific binding,
(b) screening said plurality of molecules, and
(c) detecting specific binding, thereby identifying a ligand that specifically binds to said HLA-E protein.

21. A method for detecting CD94/NKG2-positive cells comprising the steps of:
(a) supplying a sample containing cells,
(b) combining a polypeptide sequence of claim 1 to said cell under conditions to allow binding of said polypeptide sequence to said cells; and
(c) detecting said binding, wherein said binding correlates with the presence of said CD94/NKG2-positive cells in the sample.

22. A method for isolating and purifying CD94/NKG2-positive cells comprising the steps of:
(a) supplying a sample containing cells,
(b) combining a polypeptide sequence of claim 1 to said cells under conditions to allow binding of said polypeptide sequence to said cells,
(c) isolating said CD94/NKG2-positive cells in the sample; and
(d) purifying said CD94/NKG2-positive cells.

23. A method for modulating the activity of CD94/NKG2-positive cells comprising the steps of:
(a) introducing into a host cell a nucleotide sequence of claim 7,
(b) expressing said nucleotide sequence in said host cell; and
(c) modulating said activity of CD94/NKG2-positive cells.

24. A method for modulating the activity of CD94/NKG2-positive cells comprising the steps of:
(a) providing a polypeptide sequence of claim 1,
(b) administering to cells or whole animals said polypeptide sequence of claim 1,
(c) producing a response to said CD94/NKG2-positive cells; and
(d) modulating said activity of CD94/NKG2-positive cells.

25. A method for constructing major histocompatibility complex class I single chain trimers comprising the steps of:
(a) excising peptide-encoding, or HLA-E heavy-chain-encoding region of the polynucleotide molecule of claim 7 by restriction endonuclease digestion,
(b) replacing excised region with at least one peptide-encoding or heavy chain-encoding sequence,
(c) expressing encoded recombinant polypeptide in a vector,
(d) isolating said recombinant polypeptide, and
(e) purifying said recombinant polypeptide.

26. A method for constructing major histocompatibility complex class I single chain dimers comprising the steps of:
(a) excising peptide-encoding, or HLA-E heavy-chain-encoding region of the polynucleotide molecule of claim 11 by restriction endonuclease digestion,
(b) replacing excised region with at least one peptide-encoding or heavy chain-encoding sequence,
(c) expressing encoded recombinant polypeptide in a vector,
(d) isolating said recombinant polypeptide, and
(e) purifying said recombinant polypeptide.
